(19) United States
(12) Patent Application Publication
(10) Pub. No.: US 2026/0216478 A1
(43) Pub. Date: Jul. 30, 2026

(54) INJECTION SYSTEMS

(71) Applicant: Mayo Foundation for Medical Education and Research, Rochester, MN (US)

(72) Inventors: Emanuel C. Trabuco, Rochester, MN (US); Jordan D. Miller, Rochester, MN (US); Erika K. Ross, San Francisco, CA (US)

(21) Appl. No.: 19/148,780

(22) PCT Filed: Feb. 8, 2024
(86) PCT No.: PCT/US2024/015014

§ 371 Date: Jul. 17, 2025

Related U.S. Application Data
(60) Provisional application No. 63/448,183, filed on Feb. 24, 2023.

(51) Int. Cl. A61M 25/00; A61M 5/32

(52) U.S. Cl. CPC A61M 25/0084; A61M 5/3295; A61M 2025/0085; A61M 2202/0427; A61M 2205/19; A61M 2210/1089

(57) ABSTRACT

Devices and methods are described for injecting therapeutics into tissue. For example, some embodiments disclosed in this document include devices and methods for injecting muscle precursor cells into the sphincter urethrae to treat stress urinary incontinence.

### CROSS-REFERENCE TO RELATED APPLICATIONS

This application claims the benefit of U.S. Provisional Application Ser. No. 63/448,183, filed Feb. 24, 2023. The disclosure of the prior application is considered part of, and is incorporated by reference in, the disclosure of this application.

### BACKGROUND

### 1. Technical Field

This document relates to devices and methods for injecting therapeutics into tissue. For example, some embodiments disclosed in this document relate to devices and methods for injecting bulking agents, agents that promote muscle regeneration and improve muscle function, and muscle precursor cells/adult stem cells into the sphincter urethrae to treat stress urinary incontinence. Broader embodiments disclosed in this document relate to devices and methods for injecting bulking agents, agents that promote muscle regeneration and improved muscle function, and muscle precursor cells into other structures in which compromised sphincter function results in impaired physiological function (including but not limited to treatment of the gastro-esophageal sphincter for treatment of reflux and anal sphincter for the treatment of fecal incontinence).

### 2. Background Information

Urinary incontinence afflicts between 10 to 40% of adult women in the United States. Stress urinary incontinence (SUI), the involuntary loss of urine associated with physical activity due to impaired function of the urinary sphincter, is the most common form representing approximately one-third of cases. Although not life threatening, urinary incontinence greatly impacts a woman's quality of life; with similar Health Utility Index scores being reported among women seeking treatment for stress urinary incontinence (0.67-0.73) and community dwelling women with other chronic, debilitating illnesses such as stroke (0.67), cancer (0.80), diabetes mellitus (0.74) and back pain (0.80).

Sphincter dysfunction is not an uncommon root cause of a number of other medical conditions and surgical complications, and includes gastro-esophageal reflux, fecal incontinence, overactive bladder, pelvic organ prolapse, urgency incontinence, functional incontinence, overflow incontinence, rectal prolapse, achalasia, esophageal strictures, urinary incontinence in men after radical prostatectomy, and a host of other conditions.

About 3.4 million men in the United States have urinary incontinence. In men, urinary incontinence can be brought on by various medical conditions such as enlarged prostate, diabetes, and Parkinson's disease. It is also be common after some types of prostate surgery. Urinary incontinence is a treatable condition.

### SUMMARY

This document describes devices and methods for injecting therapeutics into tissue. For example, some embodiments disclosed in this document describe devices and methods for injecting bulking agents, agents that promote muscle regeneration and improve muscle function, and muscle precursor cells/adult stem cells into the sphincter urethrae to treat stress urinary incontinence. The devices and methods described herein can be used to treat both men and women.

In one aspect, an injection device is described herein. In one embodiment, the injection device includes a handle, a shaft extending distally from the handle, two or more needles, a first slider mechanism configured to control movement of the two or more needles along a longitudinal axis of the shaft; and a second slider mechanism configured to control radial movement of the two or more needles between: (i) a first position that is fully within the shaft and (ii) a second position in which a distal tip portion of each of the two or more needles extends radially from the shaft. Mechanical engagement between the first slider mechanism and second slider mechanism prevents movement of the two or more needles along a longitudinal axis of the shaft when the two or more needles are in the second position in which the distal tip portion of the needles extends radially from the shaft.

Such an injection device may optionally include one or more of the following features. The injection device may also include a safety switch rotatably coupled to the first slider mechanism. The safety switch may maintain a relative position between the first slider mechanism and the second slider mechanism when the safety switch is lowered over the second slider mechanism. The injection device may also include a ruled indicator showing radial extension positions of the two or more needles. The two or more needles may be four hypodermic needles.

The shaft may include an inner shaft and an outer shaft. The first slider mechanism may be configured to control movement of the two or more needles along the longitudinal axis of the shaft within the outer shaft. The second slider mechanism may be configured to control movement of the two or more needles along the longitudinal axis of the shaft within the inner shaft. The first slider mechanism may define a bi-leveled groove. The second slider mechanism may include a post positioned within the bi-leveled groove. Mechanical engagement between the bi-leveled groove and the post may prevents movement of the first slider mechanism when the two or more needles are in the second position. The two or more needles can be actuated to emerge radially from the shaft at multiple positions along the longitudinal axis of the shaft. The injection device may also include an indicator that shows a position of the two or more needles along the longitudinal axis of the shaft. The shaft may have a measuring scale proximal of the two or more needles. The injection device may also include an actuation indicator that indicates whether the needles are in the first position or the second position. The actuation indicator may be positioned along the handle. The actuation indicator may be positioned on a safety switch that is configured to cover the second slider mechanism. The injection device may also include a treatment fluid reservoir configured to distribute treatment fluid between the two or more needles.

In another aspect, this disclosure is directed to a method of treating urinary incontinence. In some embodiments, the method includes: inserting a shaft of an injection device into a urethra so that a distal tip portion of the shaft resides within a bladder, positioning a meatus clamp of the injection device against a urethral meatus of the urethra, after positioning the meatus clamp, measuring a length of the urethra using a scale on the shaft, sliding a first slider mechanism along a longitudinal axis of the shaft to cause two or more needles to move longitudinally along the shaft, sliding a second slider mechanism distally along the shaft to cause the two or more needles to extend radially from the shaft so that a distal tip portion of each of the two or more needles puncture and extend through an inner wall of the urethra, and while the two or more needles are extending through the inner wall of the urethra, injecting a therapeutic via the two or more needles.

Such a method may optionally include one or more of the following features. The method may include expanding an expandable member that is attached to the distal tip portion of the shaft, and applying proximal traction of the shaft to cause the expandable member to abut an inner wall of the bladder around an opening to the urethra, wherein the length of the urethra is measured while the proximal traction is being applied. Sliding the second slider mechanism distally along the shaft to cause the two or more needles to extend radially from the shaft may include: raising a safety switch to expose the second slider mechanism, sliding the second slider mechanism distally to an actuated position in which a portion of the second slider mechanism manually engages a portion of the first slider mechanism to prevent movement of the first slider mechanism, and lowering the safety switch over the second slider mechanism. The method may include, after injecting the therapeutic, sliding the second slider mechanism proximally along the longitudinal axis of the shaft so that the distal tip portion of each of the two or more needles withdraws inside the shaft. The two or more needles may be moved longitudinally along the shaft and multiple injections are delivered at multiple locations longitudinally along the urethra. The therapeutic may include exosomes.

Particular embodiments of the subject matter described in this document can be implemented to realize one or more of the following advantages. In some embodiments, the devices and methods can be used to treat stress urinary incontinence. The devices include features to help a clinician verify target locations for a series of injections of therapeutics so that the injections reach the sphincter urethrae as desired. The devices include features to enable different injection depths for different agents being delivered to different depths or layers of the urethra wall. The devices also include features that reduce the risk of injury to patients by preventing axial movement of the needles of the device while the needles are inserted into the urethra. In some embodiments, the devices can be used to provide submucosal delivery of injection agents (e.g., bulking agent injection). In some embodiments, the device is used to deliver biologics, such as acellular regenerative materials or adult stems cells, to deeper layers of the urethra along the length of the urethral sphincter muscle, which triggers satellite cell activation and allows for widespread delivery of the biologics along the entire length of the sphincter. In some embodiments, stress urinary incontinence can be treated in a minimally invasive fashion using the devices and methods provided herein. Broader embodiments disclosed in this document relate to devices and methods for injecting various substances into structures in which compromised sphincter function results in impaired physiological function in a minimally invasive fashion using devices and methods described herein. These substances for injection using embodiments disclosed herein include, but are not limited to, bulking agents, bioinjectable agents, muscle stem cells, adult stem cells, synthetic and biologic biomaterials, polyacrylamide gels (e.g., Bulkamid®), pyrolytic carbon-coated beads (e.g., Durasphere®), polydimethylsiloxane suspended in a bio-excretable polyvinylpyrrolidone (PVP) carrier gel (e.g., Macroplastique®), calcium hydroxylapatite particles and saline-based carrier gel (e.g., Coaptite™), hypertonic saline, collagen, bovine dermal collagen, poly(lactic-co-glycolic acid) (PLGA), hydrogels for exosome delivery. Embodiments disclosed in this document relate to devices and methods for injecting mesenchymal stem cells, adult stem cells, smooth cells, regenerative cell therapies for muscle injuries, biological agents with regenerative properties, such as agents that promote muscle regeneration and improved muscle function, and muscle precursor cells into other structures in which compromised sphincter function results in impaired physiological function in a minimally invasive fashion using devices and methods described herein. Such minimally invasive techniques can reduce recovery times, patient discomfort, and treatment costs.

Unless otherwise defined, all technical and scientific terms used herein have the same meaning as commonly understood by one of ordinary skill in the art to which this disclosure pertains. Although methods and materials similar or equivalent to those described herein can be used to practice the disclosure, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description herein. Other features, objects, and advantages of the disclosure will be apparent from the description and drawings, and from the claims.

### DESCRIPTION OF THE DRAWINGS

FIG. 1 depicts an example urethral drug delivery device.

FIG. 2 is a schematic diagram of a female urethra and an example urethral drug delivery device.

FIG. 3 is a schematic diagram of the urethral drug delivery device inserted into the female urethra.

FIG. 4 is a schematic diagram of the urethral drug delivery device inserted into the female urethra and a balloon member of the device in an inflated state.

FIG. 5 is a schematic diagram of urethral drug delivery device with drug delivery needles of the device in a radially extended configuration.

FIG. 6 is an enlarged view of the shaft of the urethral drug delivery device.

FIG. 7 is an exploded view of the slider control system of the urethral drug delivery device.

FIG. 8 is a schematic diagram of a positional slider of the slider control system engaging with a portion of the handle of the urethral drug delivery device.

FIG. 9 is a cross-sectional side view of the of the slider control system coupled to the handle of the urethral drug delivery device.

FIG. 10 is a cross-sectional transverse view of the slider control system coupled to the handle of the urethral drug delivery device.

FIG. 11 is a cross-sectional side view of the slider control system when the needles of the urethral drug delivery device are in an unactuated state.

FIG. 12 is an enlarged top view of a portion of the handle of the urethral drug delivery device.

FIG. 13 is a side view of a portion of the urethral drug delivery device with the safety switch of the slider control system raised.

FIG. 14 is a cross-sectional side view of the slider control system when the needles of the urethral drug delivery device are in an actuated state.

FIG. 15 is an enlarged top view of a portion of the handle of the urethral drug delivery device.

FIG. 16 is a transverse view of a shaft of the urethral drug delivery device showing a first example orientation of the drug delivery needle's orientation radially extending from the shaft.

FIG. 17 is another transverse view of the shaft of the urethral drug delivery device showing a second example orientation of the drug delivery needle's orientation radially extending from the shaft.

FIG. 18 is a schematic diagram of the urethral drug delivery device as it is injecting a therapeutic (e.g., PEP matrix, bulking agent, etc.) into the urethral sphincter muscles.

FIG. 19 is a schematic diagram of the urethral drug delivery device after contraction of the balloon member and retraction of the needles from the urethra.

FIG. 20 is a cross-sectional bottom view of the urethral drug delivery device.

FIG. 21 is a schematic diagram depicting the removal of the urethral drug delivery device from the female urethra.

FIG. 22 is a flowchart depicting a method of treating urinary incontinence using a urethral drug delivery device as described herein.

Like reference numbers represent corresponding parts throughout.

### DETAILED DESCRIPTION

This document describes devices and methods for injecting therapeutics into tissue. For example, some embodiments disclosed in this document describe devices and methods for injecting muscle precursor cells into the sphincter urethrae to treat stress urinary incontinence.

By the new devices and methods disclosed herein, the inventors are implementing innovative therapies utilizing, for example, exosomes to recruit local stem cell migration and differentiation (which is distinguished from traditional cell-based regenerative technology). The inventors have discovered that exosomes have the capability to induce local MPC cell migration and differentiation to restore external urethral sphincter function. Accordingly, this treatment offers a novel and less costly treatment option for SUI.

This disclosure describes a delivery system for any therapeutic substances (also referred to as treatment fluid herein), including bulking agents, cell based therapies, or other compounds, substrates, or therapeutic substances, without limitation. Potential substances than can be delivered using the delivery system described herein include, but are not limited to, bulking agents, bioinjectable agents, synthetic and biologic biomaterials, platelet lysate, polyacrylamide gels (e.g., Bulkamid®), pyrolytic carbon-coated beads (e.g., Durasphere®), polydimethylsiloxane suspended in a bio-excretable polyvinylpyrrolidone (PVP) carrier gel (e.g., Macroplastique®), calcium hydroxylapatite particles and saline-based carrier gel (e.g., Coaptite™), hypertonic saline, collagen, bovine dermal collagen, poly(lactic-co-glycolic acid) (PLGA), hydrogels for exosome delivery, mesenchymal stem cells, adult stem cells, smooth cells, regenerative cell therapies for muscle injuries, biological agents with regenerative properties, such as agents that promote muscle regeneration and improved muscle function, and muscle precursor cells. Additional therapeutic substances that can be delivered using the delivery system are described in Lightner, Amy L et al., “Matrix-Delivered Autologous Mesenchymal Stem Cell Therapy for Refractory Rectovaginal Crohn's Fistulas,” Inflammatory bowel diseases, Volume 25, Issue 5 (2020): 670-677; A. Dietz et. al., “A consistent, and predictable drug: The first 100 patients treated with autologous adipose derived mesenchymal stromal cells (MSCs) at the Mayo Clinic,” Cryotherapy, Volume 19, Issue 5, Supplement (2017): S155; A. B. Dietz., et. al., “Autologous Mesenchymal Stem Cells, Applied in a Bioabsorbable Matrix, for Treatment of Perianal Fistulas in Patients With Crohn's Disease,” Gastroenterology, Volume 153, Issue 1 (2017): 59-62.E2; Rolland, Tyler J et al., “Exosome biopotentiated hydrogel restores damaged skeletal muscle in a porcine model of stress urinary incontinence,” NPJ Regenerative medicine vol. 7, 1 (2022), Article 58; and Carr, Lesley K et al, “Autologous muscle derived cell therapy for stress urinary incontinence: a prospective, dose ranging study,” The Journal of urology vol. 189, 2 (2013): 595-601, which are incorporated by reference in their entirety.

In some embodiments, the delivery system can be used to measure the length of the urethra, and to deliver a total volume of therapeutic substances, such as exosome matrix, between 5 mL and 10 mL along the urethra. The delivery system is designed to cause minimal discomfort and to be used in the outpatient setting obviating the need to go to the operating room. Measuring the length of the urethra is beneficial as the external urethral sphincter is not readily visualized, and the anatomy of the urethra of an individual changes along the longitudinal axis and ventral dorsal axis of the urethra. In addition, the anatomy of an individual's urethra changes with age. As will be described in further detail herein, the delivery system can be used to deliver the therapeutic substance at one or more positions along the length of the urethra that are determined based on a measured length of the urethra.

The devices described herein have retractable hypodermic needles that are deployed at the set distance (after the measurement of length is obtained) to deliver bilateral therapeutic substance to the external urethral sphincter. As will be described in further detail herein, the delivery system is designed to prevent axial movement of the hypodermic needles when they are in a deployed (extended) position, which reduces the risk of injury to the patient.

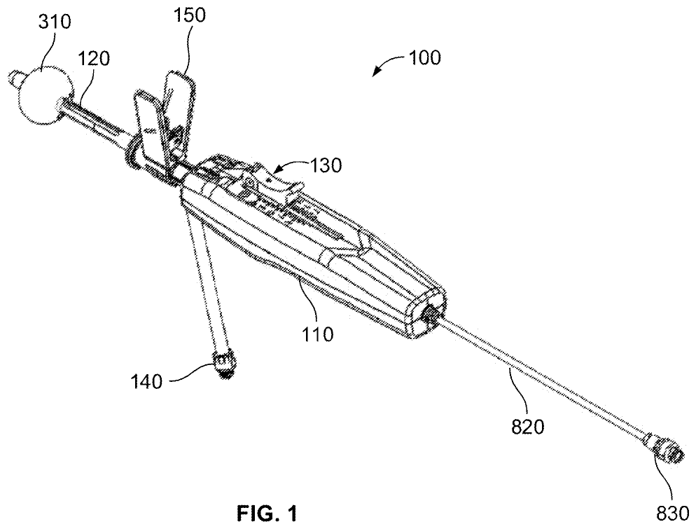

FIG. 1 shows an example urethral drug delivery device 100. The urethral drug delivery device 100 can be used by a clinician to treat urinary incontinence as described further herein. For the purposes of the descriptions herein, the components of the urethral drug delivery device 100 to the right in FIG. 1 are deemed as oriented proximal to the user (e.g., the clinician), and components to the left are deemed as oriented distally to the user.

The urethral drug delivery device 100 includes a handle 110 and a shaft 120 that distally extends from the handle 110. To perform the urinary incontinence treatment, the shaft 120 of the urethral drug delivery device 100 is designed to be inserted into the urethra 11 and partially advanced into the bladder 12. As will be described in further detail herein, the handle 110 includes a slider control system 130 for controlling movement of needles along the shaft 120 and deployment of needles through the shaft 120 into a portion of the urethra 11. The

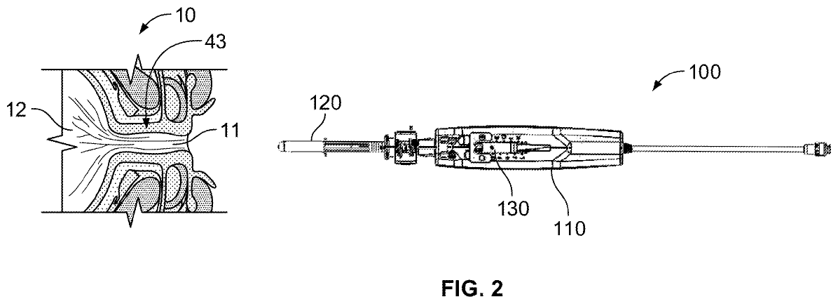

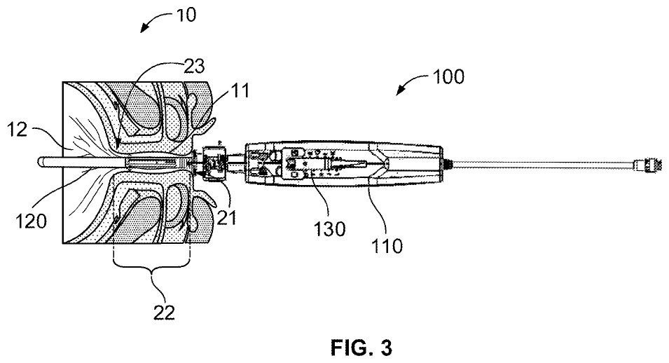

FIG. 2 shows a cross-section of a female urethral area 10 and the urethral drug delivery device 100. Referring also to FIG. 3, the shaft 120 of the urethral drug delivery device 100 can be inserted into the urethra 11. The distal blunt tip portion of shaft 120 is inserted through a urethral opening 21, along a length 22 of the urethra 11, through a neck 23 of the bladder 12, and into the bladder 12.

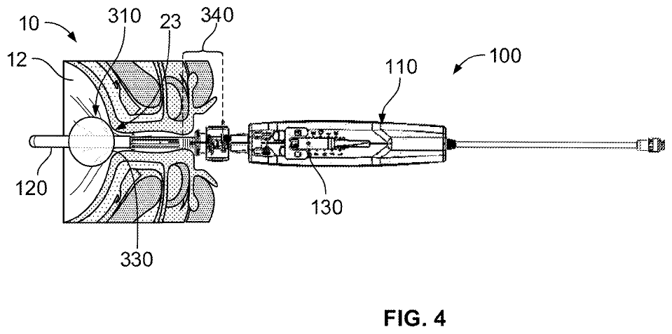

Referring to FIGS. 1 and 4, an expandable balloon 310 may be inflated within the bladder 12. The balloon 310 is attached to the distal tip portion of the shaft 120. The expandable balloon 310 is inflated using a fluid provided through a balloon inflation port 140 that can be coupled to the handle 110. The balloon inflation port 140 can be used to direct fluid through the luminal portion of the shaft 120 and cause the expandable balloon 310 to inflate. In some embodiments, a mechanical expendable element is used instead of the balloon 310.

With the balloon 310 in its expanded state, the urethral drug delivery device 100 may then be held in place by the clinician with light tension applied via the handle 110 in a proximal direction such that the proximal base 330 of the expandable balloon 310 is abutting the neck 23 of the bladder 12. Accordingly, the balloon 310 can be used in this manner to positively register the position of the urethral drug delivery device 100 in relation to the female urethral area 10. Once the urethral drug delivery device 100 is correctly positioned with the proximal base 330 of the expandable balloon 310 is abutting the neck 23 of the bladder 12, a meatus clamp 150 is advance forward to the urethral meatus. The meatus clamp 150 helps ensures that the shaft 120 of the device cannot be pushed further into the patient's bladder after the device 100 has been positioned and the balloon 310 has been expanded

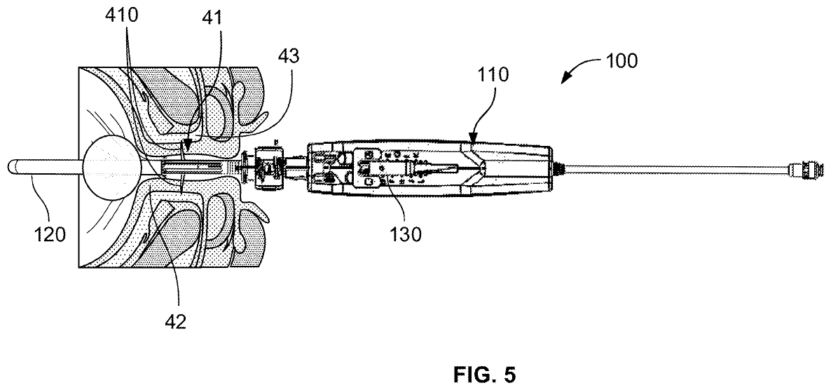

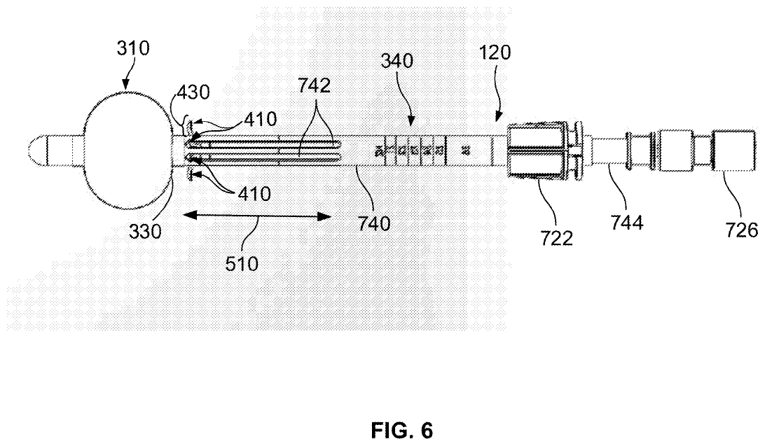

Referring to FIGS. 5 and 6, with the balloon 310 held against the neck 23 of the bladder 12 and the meatus clamp 150 advanced so that it abuts the meatus, the length 22 of the urethra 11 is measured by the clinician using a section of ruled markings 340 on the shaft 120. The average length of the female urethra is approximately 4 cm±1 cm and the ruled markings 340 may be positioned proximally along the shaft 120 from the base 330 of the expandable balloon 310 such that this range (e.g., ≥3 cm, ≤5 cm) can be visualized in millimeter increments. In some embodiments, the ruled markings 340 on the shaft 120 are located approximately 63.5 mm to 93.5 mm proximal from the base 330 of the expandable balloon 310. Measuring the length 22 of the urethra 11 is performed by the clinician to determine the length of the urethra 11. As will be described in further detail herein, the number of injections and the one or more positions along the length of the urethra that the needles of the device should be deployed and the injections provided is determined based on the measured length 22 of the urethra 11

Referring also to FIGS. 5 and 6, the urethral drug delivery device 100 includes two or more injection needles 410 that can be used to deliver a therapeutic substance. The clinician can control movement of the needles 410 at along the longitudinal axis of the shaft 120, as well as control the needles 410 to radially extend from the shaft 120 or be contained within the shaft 120. As can be seen in FIGS. 3 and 4, as the shaft 120 is advanced into the urethra 11, the needles 410 are kept contained within the shaft 120. Thereafter, the needles 410 can be actuated by the clinician using the slider control system 130 to radially extend outside the shaft 120 (so as to puncture the surface of the tissue of the urethra 11, and to extend into a urethral sphincter 43 around the urethra 11).

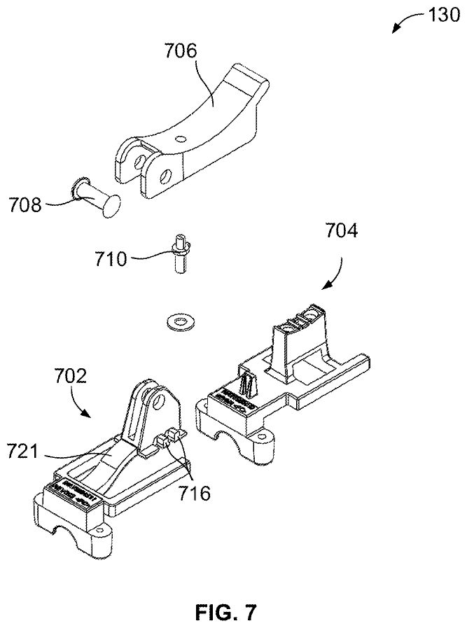

The slider control system 130 can be used to control movement of the needles 410 along the longitudinal axis of the shaft 120, as well as to control the radially extension and retraction of the needles 410 from the shaft 120. Referring to FIG. 7, the slider control system 130 includes a positional slider 702, an actuation slider 704, and a safety switch 706. The safety switch 706 is rotatably coupled to the positional slider 702 using a pin 708 that is inserted through corresponding opening in the safety switch 706 and the positional slider 702.

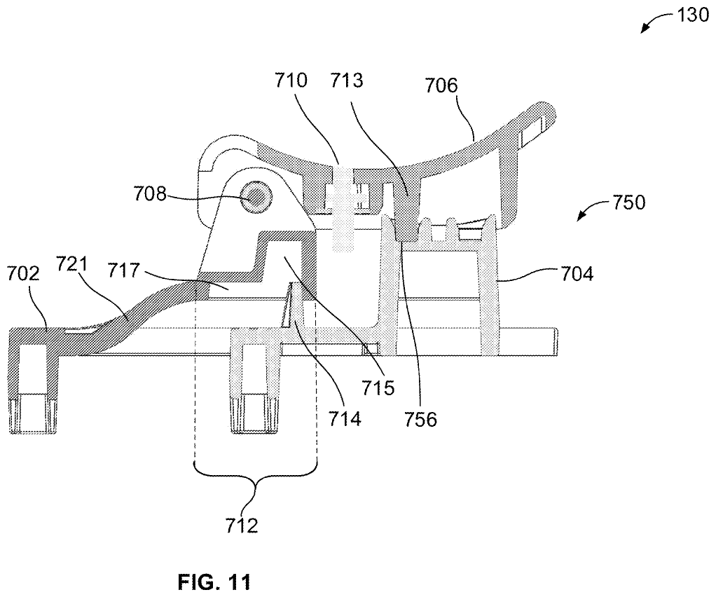

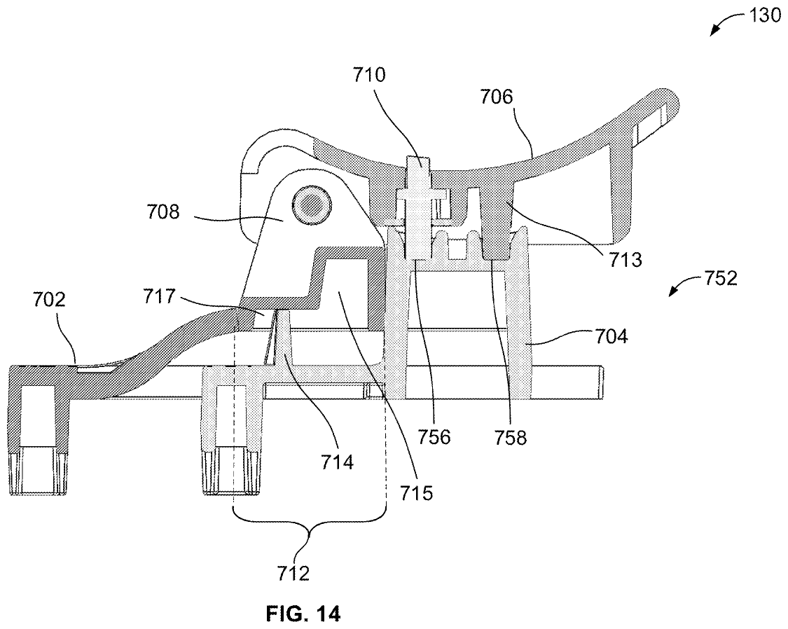

The slider control system 130 also includes an actuation indicator 710 that indicates to a clinician whether the needles 410 are extended radially outside the shaft 120. As can be seen in FIG. 11, the actuation indicator is coupled to the safety switch 706 with a retention washer 711. As will be discussed in further detail herein, when the needles 410 are actuated to extend outside the shaft 120, the actuation indicator interacts with a groove 703 on an upper surface of the actuation slider 704 and is raised proud to the upper surface of the safety switch 706, as depicted in FIG. 14. When the needles are unactuated and positioned inside the shaft 120, the retention washer 711 contacts the actuation indicator 710 and positions the top of the actuation indicator 710 flush with the upper surface of the safety switch. By feeling whether the actuation indicator 710 is flush with or proud to the surface of the safety switch 706, a clinician can quickly and easily determine whether the needles 410 are actuated outside the shaft 120 (e.g., into the tissue of the urethra 11).

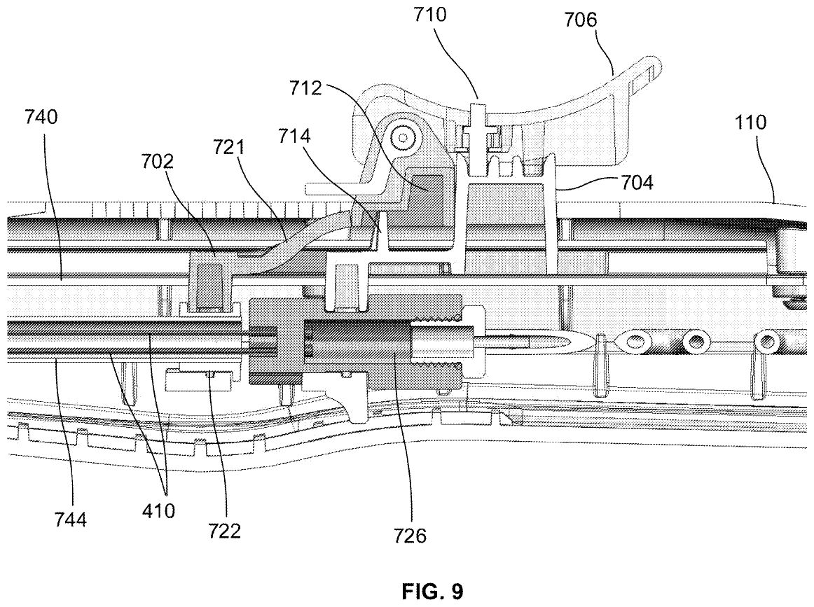

As can be seen in FIGS. 9 and 11, the positional slider 702 defines a bi-level groove 712 on a bottom portion the positional slider 702, and the positional slider is mechanically coupled to the actuation slider 704 through cooperation between a post 714 extending from an upper surface of the actuation slider 704 and the bi-level groove 712. As will be described in further detail herein, the interaction between the bi-leveled groove 712 of the positional slider 702 and the post 714 of the actuation slider prevents movement of the positional slider 702 when the needles 410 are extended outside the shaft 120 of the device 100.

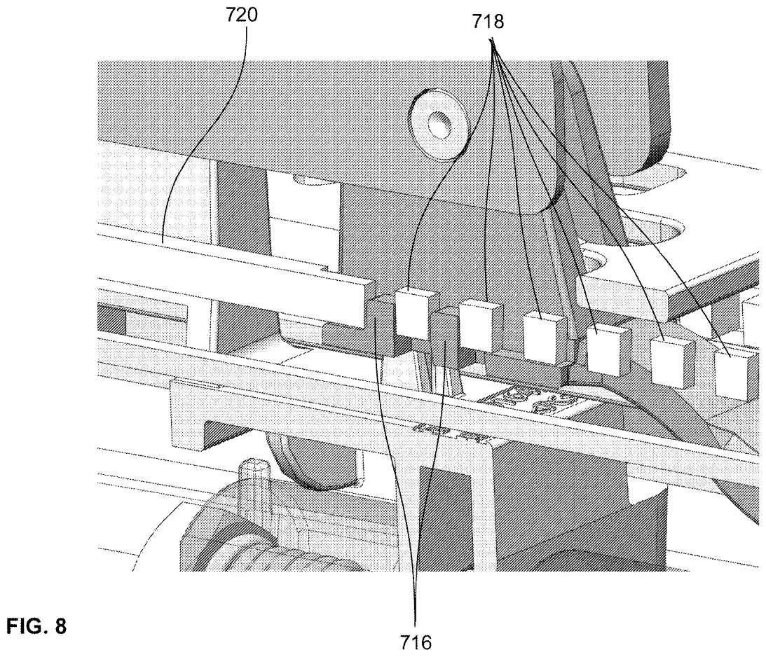

Referring to FIGS. 7 and 8, the positional slider 702 is mechanically coupled to the handle 110 of the device 100 through cooperation between teeth 716 on the positional slider 702 with corresponding locking tabs 718 defined along a rail 720 inside the handle 110. As can be seen in FIG. 8, the teeth 716 of the positional slider 702 can each be positioned between respective adjacent locking tabs 718 along the rail 720, which prevents movement of the positional slider 702 along the longitudinal axis of the handle 110. In order to move the positional slider 702 along the longitudinal axis of the handle 110, a clinician can apply a downward force on the positional slider 702 (e.g., by pressing downward on the safety switch 706), and the downward force is transferred to the positional slider 702 through the connection of the safety switch 706 and positional slider 702 through the pin 708. The downward force applied to the positional slider 702 causes the plastic spring 721 of the positional slider 702 to flex and lower the teeth 716 of the positional slider 702 below the rail 720, unlocking the positional slider 702 from the rail 720 and enabling the positional slider 702 to move freely along the longitudinal axis of the handle 110. Once the user releases the downward force on the positional slider 702, the teeth 716 of the positional slider 702 lift upwards between the nearest two respective locking tabs 718 along the rail 720, which locks the positional slider 702 in the corresponding position along the longitudinal axis of the handle 110.

Referring to FIGS. 6 and 9, the positional slider 702 is coupled to a catheter hub 722, and each of the needles 410 is coupled to the catheter hub 722. In some implementations, the needles 410 are attached to the catheter hub 722 using a collar positioned over the base of the needles 410 and the catheter hub 722. In some implementations, the needles 410 are attached to the catheter hub 722 using an epoxy substance. Movement of the positional slider 702 along the longitudinal axis of the handle 110 causes the catheter hub 722 and needles 410 to move a corresponding distance and direction along the longitudinal axis of the handle 110 within an outer catheter 740 of the shaft 120. As a result, sliding the positional slider 702 along the longitudinal axis of the handle 110 causes corresponding axial movement of the needles 410 within the outer catheter 740, allowing a clinician to move the needles 410 to a particular position within the urethra 11 for providing an injection at the respective position.

As can be seen in FIG. 11, when the actuation slider 704 of the slider control system is in an unactuated position 750, the post 714 of the actuation slider 704 is positioned beneath a taller portion 715 of the bi-leveled groove 712, which enables the spring 721 of the positional slider 702 to be pressed downward to allow for axially travel of the positional slider 702 along the handle 110. In addition, when the actuation slider 704 of the slider control system is in an unactuated position 750 and the safety switch 706 is in a lowered position over the actuation slider 704, a post 713 on the safety switch 706 engages with a groove 756 on the upper surface of the actuation slider 704. The engagement between the post 713 of the safety switch 706 and groove 756 of the actuation slider 704 helps to maintain the relative positioning between the positional slider 702 and the actuation slider 704. As a result, the actuation slider 704 moves together with the position slider 702 axially along the handle 110 when the actuation slider 704 is in the unactuated position 750, which ensures that the actuation slider 704 does not move into an actuated position 752 (as depicted in FIG. 14) and that the needles 410 are not extended out of the shaft 120 during movement of the positional slider 702 along the longitudinal axis of the device 100.

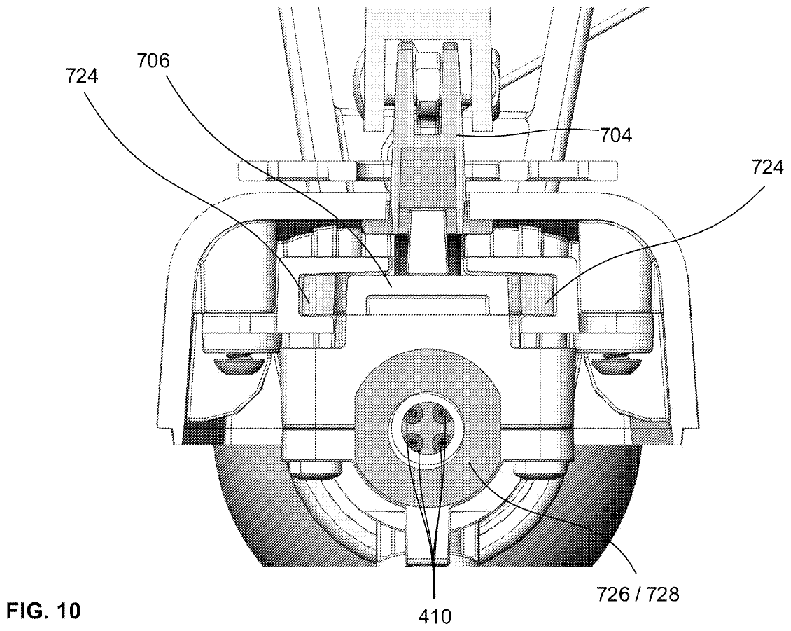

Referring to FIGS. 6 and 10, the actuation slider 704 is coupled to a pair of rails 724 within the handle 110 and can slide along the longitudinal axis of the handle 110 along the rails 724. The actuation slider 704 is coupled to a treatment fluid reservoir 726 located within an inner catheter 744 nested within the outer catheter 740, and a proximal end of each of the needles 410 is coupled to the treatment fluid reservoir 726. As a result, when a clinician slides the actuation slider 704 distally along the handle 110, the treatment fluid reservoir 726 also moves distally within the inner catheter 744 and applies a distal force to the ends of the needles 410. The distal force applied to the ends of the needles 410 by the actuation slider 704 causes the distal tip of each of the needles 410 to abut a distal end of the inner catheter 744 and extend radially outside the shaft 120 through a needle guide tip of the inner catheter 744 and through slots 742 defined in the outer catheter 740. Conversely, when the needles 410 are in a radially extended position, the clinician can slide the actuation slider 704 proximally along the handle 110 to cause the treatment fluid reservoir 726 to move proximally within the inner catheter 744 and apply a proximal force to the ends of the needles 410 to cause the needles 410 to withdraw into the shaft 120. In some implementations, the ends of the needles are welded to a washer 728 of the treatment fluid reservoir 726. As will be discussed in further detail herein, the treatment fluid reservoir 726 is configured to receive a treatment fluid from an injection port 830 and distribute the treatment fluid to the needles 410 for injection into the urethra 11.

A process for positioning and controlling the needles 410 to administer one or more injections of a treatment fluid using the urethral drug delivery device 100 will now be described in reference to FIGS. 5-20.

As previously described, the shaft 120 is inserted into the urethra 11 with the needles 410 in an unactuated position (i.e., contained within the shaft 120). A measurement of the length 22 of the urethra 11 is obtained using the ruled markings 340. Based on the measured length 22 of the urethra 11, the clinician can determine the number of injections to be performed and a corresponding position along the length of the urethra 11 for performing each injection. For example, in some implementations, the clinician references a manual or guide (e.g., a table) that specifies the appropriate number of injections to be performed based on the length 22 of the urethra 11 and the appropriate axial position to place the needles 410 for each of the injections based on the length 22 of the urethra 11.

In some implementation, the needles 410 will be deployed and injections will be provided in 3 to 5 axial positions along the urethra 11 based on the length 22 of the urethra 11. As an example, if a patient's urethra 11 is measured to be 3.2 cm long, the clinician can determined that 0.42 mL of treatment fluid should be provided through each of the needles 410 of the device 100 at three distinct axial positions along the patient's urethral sphincter 43, which results in a total of 5 mL of treatment fluid being provided to the patient's urethral sphincter 43.

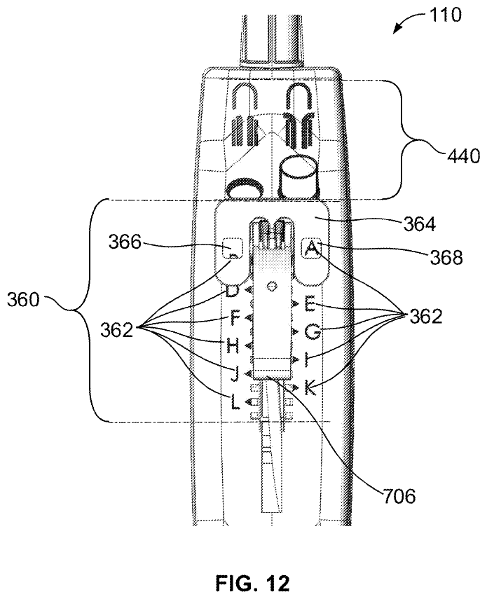

Once the length 22 of the urethra 11 has been measured using the ruled markings 340 and the number of injections and respective position for each injection has been determined based on the measured length 22, the position of the needles 410 along the longitudinal axis of the shaft 120 may be adjusted by the clinician using the positional slider 702 until the needles 410 are positioned at the correct axial position for the first injection. Referring to FIG. 12, the device includes an axial location indicator 360 that indicates the axial location of the needles 410 along the longitudinal axis of the shaft 120 and can be used by the clinician to properly position the needles 410 at the one or more injection positions that are determined based on the measured length 22 of the urethra 11. The axial location indicator 360 may be ruled, for example, in millimeter increments. In the depicted embodiments, the axial location indicator 360 includes multiple markings 362 on the top surface of the handle that each correspond to a particular axial position of the needles 410. For example, the axial location indicator 360 may have a first marking 362 (“A”) that denotes the maximal distal position of the needles 410 in the urethra 11, a second marking 362 (“L”) proximal of the first marking denoting the maximal proximal position of the needles 410 in the urethra 11, and one or more additional markings 362 (“B”-“K”) between the first and second markings 362 denoting additional discrete positions of the needles 410 along the urethra 11. As described above, in some implementations, the clinician can use a guide or manual to determine which of the positions A-L the needles 410 should be placed for injections based on the measured length 22 of the urethra 11.

The axial location indicator 360 also includes a current position indicator 364 that is mechanically coupled to positional slider 702 and can be used by a clinician to determine the current position of the needles 410 within the urethra 11. The current position indicator 364 defines viewing windows 366, 368 on either side of the positional slider 702, and, as the positional slider 702 is moved along the longitudinal axis of the handle 110, the marking 362 indicating the current axial position of the needles 410 is positioned within and visible through one of the viewing windows 366, 368. A clinician can confirm correct placement of the needles 410 at a particular axial position within the urethra 11 by confirming that the marking corresponding to the desired positioning of the needles 410 is visible through one of the viewing windows 366, 368.

As previously described, in order move the needles 410 along the longitudinal axis of the shaft 120, a clinician can press downward on the safety switch 706 to apply a downward force on the positional slider 702, which causes the plastic spring 721 of the positional slider 702 to flex and lowers the teeth 716 of the positional slider 702 below the rail 720 inside the handle, enabling the positional slider 702 to move freely along the longitudinal axis of the handle 110. As previously discussed, the engagement between the post 713 of the safety switch 706 and the groove 756 of the actuation slider 704 when the actuation slider 704 is in the unactuated position 750 causes the actuation slider 704 to move together with the position slider 702 axially along the handle 110 when the actuation slider 704 is in the unactuated position 750 and prevents movement of the actuation slider 704 into an actuated position 752 (as depicted in FIG. 14) during longitudinal movement of the positional slider 702. As a result, actuation of the needles 410 out of the shaft 120 of the device 100 is prevented during axially positioning of the needles 410 using the positional slider 702.

Referring to FIG. 6, in some embodiments, the axial position of the needles 410 within the outer catheter 740 along the longitudinal axis of the shaft 120 may be adjusted by the clinician across an axial distance range 510 of 5 mm to 43 mm as measured from the from the proximal base 330 of the expandable balloon 310 (to provide a range of needle advancement locations into the urethral sphincter 43). In some implementations, the needles 410 can be moved along the longitudinal axis of the shaft 120 in increments of 3.5 mm. The axial location indicator 360 may be ruled to cover the complete axial range 510. The outer catheter 740 defines a plurality of slots 742 along the axial range 510, and each of the needles 410 can be actuated to extend radially from the outer catheter 740 through a respective slot 742 at various positions along the axial range 510.

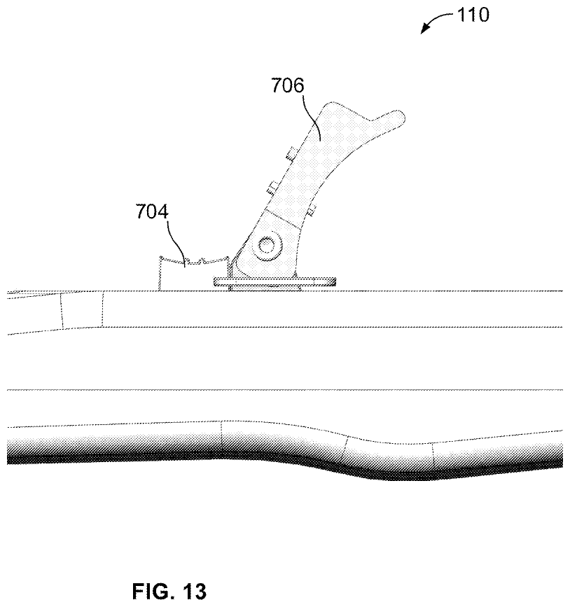

Once the clinician has confirmed that the needles 410 are at the desired position within the urethra 11 using the axial location indicator 360, the clinician releases the downward force on the safety switch 706, which causes the teeth 716 of the positional slider 702 to lift upwards between the nearest two respective locking tabs 718 of the rail 720 within the handle 110, which locks the positional slider 702 in place along the longitudinal axis of the handle 110. Once the needles 410 are positioned at the desired location within the urethra 11, the clinician may then cause the needles 410 to be radially extended from the walls of the shaft 120 using the actuation slider 704. For example, as depicted in FIG. 13, the clinician can rotate the safety switch 706 upwards to expose the actuation slider 704. Once the actuation slider 704 is exposed, the clinician can slide the actuation slider 704 distally along the handle 110 to an actuated position, which causes the treatment fluid reservoir 726 coupled to the actuation slider 704 to move distally within the inner catheter 744 and apply a distal force to the ends of the needles 410. The distal force applied to the ends of the needles 410 by the actuation slider 704 causes the distal tips of the needles 410 to abut an end of the inner catheter 744 and extend radially outside the shaft 120 through openings in the inner catheter 744 and respective slots 742 defined in the outer catheter 740, and bilaterally advances the needles 410 through the mucosa 41 and smooth muscle 42 of the urethra 11 and into the urethral sphincter 43.

The actuation slider 704 may be used by the clinician to control the radial extension 430 of the needles 410 between 8.65 mm and 9.35 mm outside of the shaft 120. In some embodiments, the needles 410 are formed of nitinol and automatically conform to a predetermined shape upon extension outside of the shaft 120. For example, as depicted in FIG. 6, the needles 410 can conform to a curved shape upon extending outside the shaft 120.

FIG. 14 depicts the slider control system 130 when the actuation slider 704 is in the actuated position, causing the needles 410 to be actuated outside the shaft 120. When the needles 410 are actuated outside the shaft 120 of the device 100, the slider control system 130 prevents the needles 410 from being moved longitudinally in order to prevent injury to the urethra 11. When the actuation slider 704 moves from the unactuated position 750 (depicted in FIG. 11) to the actuated position 752 (depicted in FIG. 14), the post 714 of the actuation slider 704 moves from being positioned beneath a taller portion 715 of the bi-leveled groove 712 to being positioned beneath a shorter portion 717 of the bi-leveled groove 712 and abutting the positional slider 702. When the actuation slider 704 is in the unactuated position 750, the post 714 of the actuation slider 704 is positioned beneath a taller portion 715 of the bi-leveled groove 712, the spring 721 of the positional slider 702 can be pressed downward to enable travel of the positional slider 702 along the longitudinal axis of the handle 110, as described herein. In contrast, when the actuation slider 704 is in the actuated position 752, the post 714 of the actuation slider 704 is positioned beneath a shorter portion 717 of the bi-leveled groove 712, and contact between the shorter portion 717 of the bi-leveled groove 712 and the top of the post 714 of the actuation slider 704 prevents the positional slider 702 from being pressed downward. Without being able to flex the spring 721 of the positional slider 702 downward, the teeth 716 of the positional slider 702 cannot be disengaged from the locking tabs 718 inside the handle 110, which prevents movement of the positional slider 702 along the handle 110. As a result, the positional slider 702 cannot be moved along the handle 110 while the needles 410 are actuated outside the shaft 120.

Once the clinician has moved the actuation slider 704 into the actuated position 752 with the post 714 of the actuation slider 704 abutting the positional slider 702, the clinician can rotate the safety switch 706 downward to cover the actuation slider 704, which maintains the relative position between the positional slider 702 and the actuation slider 704 while the needles 410 are extended. As can be seen in FIG. 14, when the actuation slider 704 is in the actuated position 752 and the safety switch 706 is lowered over the actuation slider 704, the actuation indicator 710 engages with a first groove 756 on the surface of the actuation slider 704 and the post 713 of the safety switch 706 engages with a second groove 758 on the surface of the actuation slider 704. Engagement between the actuation indicator 710 and the first groove 756 and between the post 713 of the safety switch 706 and the second groove 758 further prevents axial movement of the positional slider 702 coupled to the safety switch 706. In addition, the engagement between the actuation indicator 710 groove 756 on the surface of the actuation slider 704 causes the actuation indicator 710 to be raised above the upper surface of the safety switch 706, which provides a visual and tactile indication to the clinician that the needles 410 are actuated. In addition, lowering the safety switch 706 to cause engagement between the actuation indicator 710 and the first groove 756 and between the post 713 of the safety switch 706 and the second groove 758 helps ensure that the needles 410 are in the fully radially extended (actuated) position when the actuation slider 704 is in the actuated position 752. FIG. 6 depicts the at least two needles 410 (e.g., 2, 4, etc.) in their radially-extended configuration (i.e., being extended from the shaft by a distance of the radial extension 430).

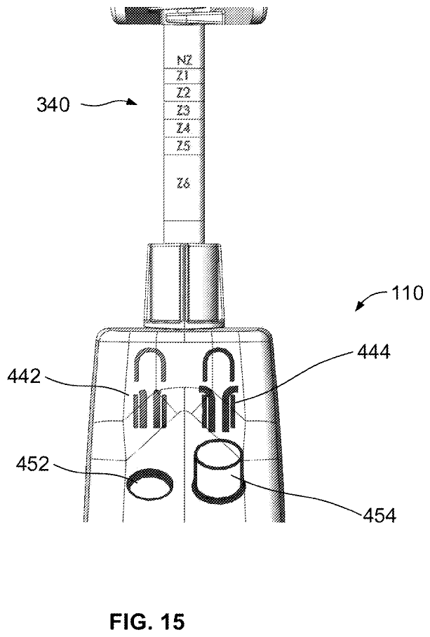

Referring to FIG. 15, the actuation indicator 440 on the handle 110 includes markings 442, 444, 452, 454 that provide a visual representation of the position of the actuation indicator 710 based on the actuation state of the needles 410. For example, the first pair of marking 442, 452 indicates that the actuation indicator 710 is flush with or below the top surface of the safety switch 706 when the needles 410 are withdrawn into the shaft 120 in the unactuated position. The second pair of markings 444, 454 indicates that the actuation indicator 710 is extends above the top surface of the safety switch 706 when the needles 410 are extended outside the shaft 120 in the actuated position. In some implementations, the markings 442, 444, 452, 454 are printed on the handle 110 of the device 100.

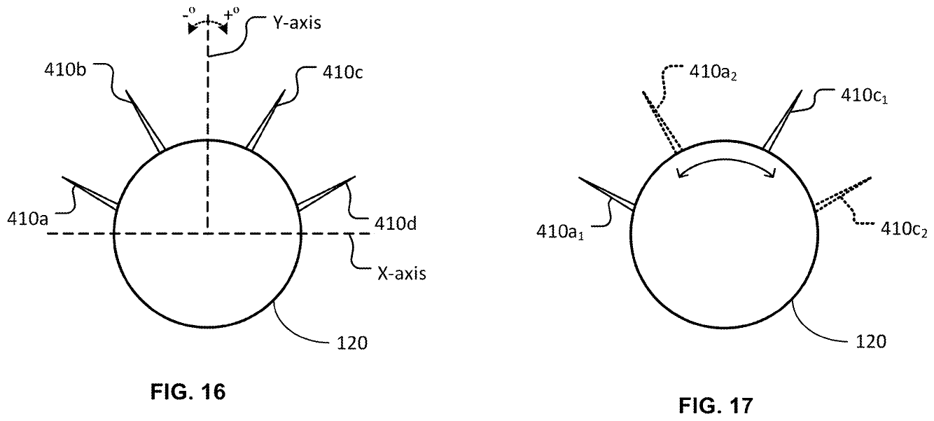

FIG. 16 shows a transverse cross-sectional view of an example embodiment of the shaft 120 and the needles 410. The depicted embodiment includes four needles 410 (indicated individually here as 410a, 410b, 410c, and 410d). While the needles 410 can be located so as to radially extend from the shaft 120 at any locations around the transverse circumference of the shaft 120, in the depicted embodiment all four needles 410 are within a 210° envelop. More particularly, in the depicted embodiment a first needle 410a is located at about −104.9° relative to the Y-axis. A second needle 410b is located at about −14.9° relative to the Y-axis. A third needle 410c is located at about 14.9° relative to the Y-axis. A fourth needle 410d is located at about 104.9° relative to the Y-axis. Hence, the total arc between the first needle 410a and the fourth needle 410d is about 209.8°.

FIG. 17 shows a transverse cross-sectional view of another example embodiment of the shaft 120 and the needles 410. The depicted embodiment includes two needles 410, that is: (i) a first needle 410a that can be positioned in a first location 410a1 and a second location 410a2, and (ii) a second needle 410c that can be positioned in a first location 410c1 and a second location 410c2. In the depicted embodiment, the first needle 410a and the second needle 410c are separated by an arc of about 90°.

In one example usage technique, the two needles 410a and 410c are actuated to radially extend into their first respective locations 410a1 and 410c1. Injections of a treatment fluid via the needles 410a and 410c can be then delivered. Thereafter, the needles 410a and 410c can be radially withdrawn back into the confines of the shaft 120 by withdrawing the actuation slider 704 into the unactuated position. Next, the clinician can rotate the shaft 120 by about 30°. In that position, the clinician can use the actuation slider 704 to cause the needles 410a and 410c to radially extend into their second respective locations 410a2 and 410c2. Then, while the needles 410a and 410c are in their radially extended configurations, second injections of the treatment fluid can be delivered. Accordingly, it can be envisioned that the embodiment of FIG. 17 (having just the two needles 410a and 410c) can be used in a two-step injection process to deliver the same four injections that the embodiment of FIG. 16 can deliver in a single injection step (and without having to rotate the shaft 120).

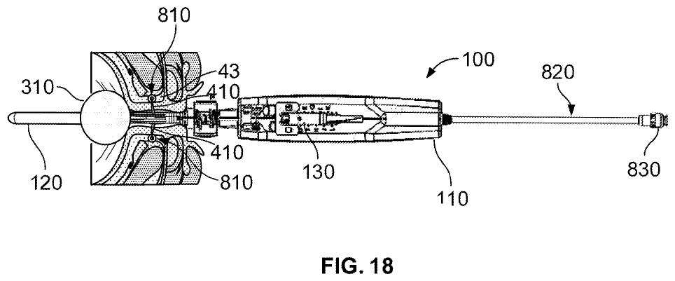

FIG. 18 shows an example injection of a treatment fluid 810 (e.g., exosome matrix) into the urethral sphincter 43. After the expandable balloon 310 has been inflated and a urethral length 22 measurement taken using the ruled markings 340, the needles 410 may be actuated to extend radially from the shaft 120 (using the actuation slider 704) to enter into the urethral sphincter 43. Upon advancing to a desired position within the radial extension 430 range (e.g., 1 cm, 2 cm, 3 cm, 4 cm, 5 cm, etc.), an injection of the treatment fluid 810 (e.g., exosome matrix) may then be administered.

Extending from the proximal end of the handle 110 is a delivery tube 820 that continues longitudinally through the body of the handle 110, and terminates at the treatment fluid reservoir 726 (as depicted in FIGS. 6 and 9). Connecting to the proximal end of the delivery tube 820 is an injection port 830 that is configured to couple to a fluid source (e.g., a syringe), and treatment fluid 810 (e.g., exosome matrix) is provided from the fluid source to the treatment fluid reservoir 726 through the injection port 830 and delivery tube 820. The treatment fluid 810 delivered to the treatment fluid reservoir 726 is distributed evenly to and flows through the needles 410 coupled to the treatment fluid reservoir 726. In some embodiments, a preferred total volume (V_{T}) of treatment fluid 810 administered may be between 5 mL and 10 mL (e.g. 10 mL≥V_{T}≥5 mL), but any other desired volumes, such as between 5 mL and 1000 mL (e.g., 1000 mL≥V_{T}≥5 mL), may also be administered.

After advancing the needles 410 and administering a first injection of the treatment fluid 810 at a desired depth within the urethral sphincter 43, the needles 410 may then be retracted into the shaft 120 by raising the safety switch 706 to expose the actuation slider 704 and sliding the actuation slider 704 proximally into the unactuated position 750, as depicted in FIG. 11. Once the actuation slider 704 is returned to the unactuated position 750, the safety switch 706 is lowered over the actuation slider 704 to fix the relative position of the positional slider 702 and actuation slider 704 via engagement between post 713 and groove 756.

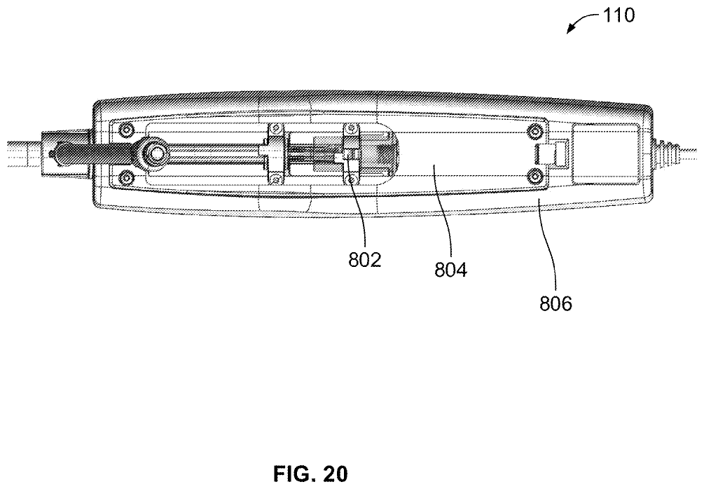

Referring to FIG. 20, in some embodiments, the device 100 includes a secondary retraction mechanism 802 that is mechanically coupled to the needles 410 and can be used by a clinician to manually withdrawn the needles 410 from an extended position back into the shaft 120 in case of failure of the actuation slider 704 (e.g., if the actuation slider 704 becomes stuck in the actuated position 752). In case of failure of the actuation slider 704, a clinician can remove a removable cover 804 on the back surface 806 of the handle to access the secondary retraction mechanism 802 and can engage the secondary retraction mechanism 802 to manually withdraw the needles 410 out of the tissue of the urethra 11 and back into the shaft 120. Once the needles 410 are withdrawn into the shaft 120, the cover 804 can be replaced by the clinician to cover the secondary retraction mechanism 802.

If a second injection is desired, once the actuation slider 704 is returned to the unactuated position 750, the needles 410 may be moved longitudinally along the shaft 120 to a new position along the urethral length 22 by actuating the positional slider 702, as described herein. Then, the needles 410 can be re-extended into the urethral sphincter 43 using the actuation slider 704 and a second injection can be administered through the needles. Thereafter, the needles 410 can again retracted into the shaft 120. This process may be repeated up to four or more times (e.g., 1, 2, 3, 4, 5, 6, etc.) at unique sites located axially along the urethral length 22. In some embodiments, the injections may be separated by spacings of between 5 mm and 10 mm apart, or 2 mm and 6 mm apart, without limitation.

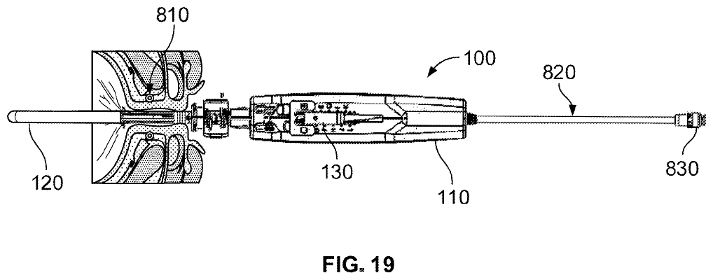

Referring also to FIG. 19, once the injection, or series of injections, is complete, the expandable balloon 310 may be deflated by removing the inflating liquid from the expandable balloon 310 through the balloon inflation port 140. For example, a clinician can attached a syringe to the inflation port 140 of the device and withdraw the inflation fluid from the balloon 310 into the syringe through the inflation port 140.

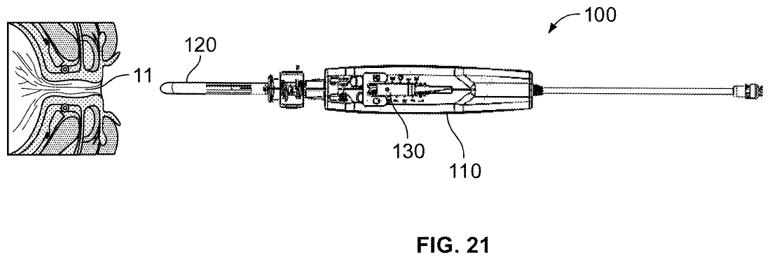

FIG. 21 shows the urethral drug delivery device 100 after removal from the urethra 11 by pulling the handle 110 proximally until the urethral drug delivery device 100 is fully clear of the urethra 11.

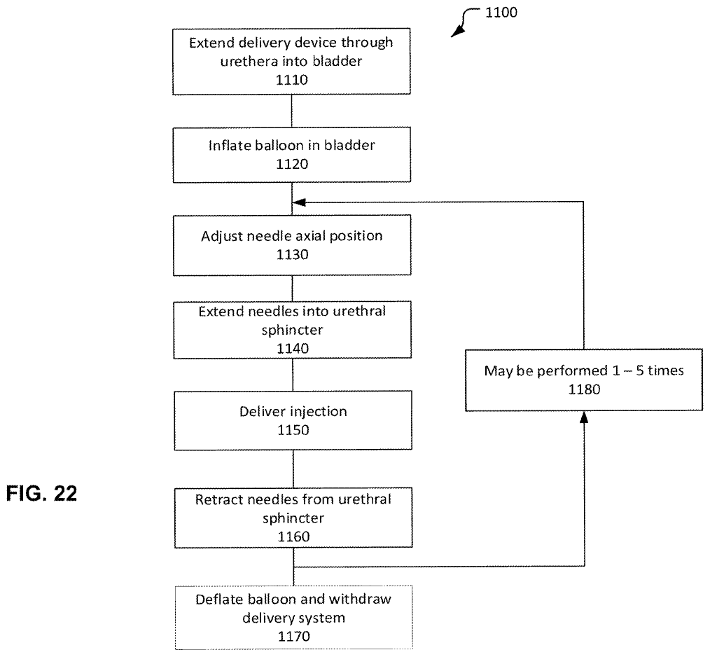

FIG. 22 is a flow diagram of a method 1100 detailing the steps 1110 through 1180 a clinician user may go through to administer one or more injections of a treatment fluid using the urethral drug delivery device 100. The steps 1110 through 1180 of the method 1100 are described in detail in reference to FIGS. 1-21.

### ADDITIONAL FEATURES, EMBODIMENTS, AND IMPLEMENTATIONS

The general concepts described herein in reference to FIGS. 1 through 22 can be used to describe broader applications of the inventive aspects to other uses/tissues. For example;

FIG. 2 could be a schematic of a tubular structure with an externally accessible orifice and specified region of desired substrate delivery.

FIG. 3 could be a schematic of the device inserted into the tubular structure.

FIG. 4 could show the inflation of the balloon at a distal point where there is a structure that enables appropriate stabilization of the balloon and confirmation of positioning.

FIG. 5 would depict the placement of the delivery of the device with the needle orientation extending radially into the approximate region of the sphincter.

FIG. 6 could be an enlarged view of the shaft of the device at the expected distance of the sphincter.

FIG. 16 could be a transverse view of a shaft of the drug delivery device showing a first example orientation of the drug delivery needle's orientation radially extending from the shaft.

FIG. 18 could be a schematic diagram of the drug delivery device as it is injecting a therapeutic (e.g., exosome matrix, bulking agent, etc . . . ) into the site of the dysfunctional sphincter muscles.

FIG. 19 could be a schematic diagram of the drug delivery device after contraction of the balloon member.

FIG. 21 could be a schematic diagram depicting the removal of the drug delivery device by retraction out of the orifice into which entry/access was enabled.

FIG. 22 could be a flowchart depicting a method of treating sphincter dysfunction using a device as described herein.

Further, while the devices and methods disclosed herein are described in the context of trans-urethral injections to treat female urinary incontinence, it should be understood that the devices and methods (or minor modifications thereof) can be implemented in many other contexts with beneficial efficaciousness. For example, in some embodiments the devices and methods described herein can be implemented for treating rectal sphincters, esophageal sphincters, ileocecal sphincters, peri-intestinal structures, perivascular structures, male incontinence, and in other contexts in which injections of therapeutic substrates are beneficial. The devices, systems, and methods described herein can be used to treat conditions such as, but not limited to, gastro-esophageal reflux, fecal incontinence, peri-vascular diseases (including malformations and other conditions), spontaneous male urinary incontinence, post-operative male urinary incontinence (e.g., following radical prostatectomy), overactive bladder, pelvic organ prolapse, urgency incontinence, functional incontinence, overflow incontinence, rectal, achalasia, esophageal strictures, and esophageal cancer (e.g., by delivering targeted anti-cancer treatments to specified regions of the esophagus).

Certain features may be increased or decreased in size or characteristics (in proportion to one another or out of proportion to one another), including but not limited to the shaft diameter, balloon size, shaft length, needle size (including but not limited to length, number of needles, outer diameter, curvature, and lumen diameter) to obtain optimal therapeutic outcomes with any variety of therapeutic substrates applied or tissues/structures targeted.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. An injection device comprising:
a handle;
a shaft extending distally from the handle;
two or more needles;
a first slider mechanism configured to control movement of the two or more needles along a longitudinal axis of the shaft; and
a second slider mechanism configured to control radial movement of the two or more needles between: (i) a first position that is fully within the shaft and (ii) a second position in which a distal tip portion of each of the two or more needles extends radially from the shaft, wherein mechanical engagement between the first slider mechanism and second slider mechanism prevents movement of the two or more needles along a longitudinal axis of the shaft when the two or more needles are in the second position in which the distal tip portion of the needles extends radially from the shaft.

2. The device of claim 1, further comprising a safety switch rotatably coupled to the first slider mechanism.

3. The device of claim 2, wherein the safety switch maintains a relative position between the first slider mechanism and the second slider mechanism when the safety switch is lowered over the second slider mechanism.

4. The device of claim 1, further comprising a ruled indicator showing radial extension positions of the two or more needles.

5. The device of claim 1, wherein the two or more needles comprises four hypodermic needles.

6. The device of claim 1, wherein:
the shaft comprises an inner shaft and an outer shaft;
the first slider mechanism is configured to control movement of the two or more needles along the longitudinal axis of the shaft within the outer shaft; and
the second slider mechanism is configured to control movement of the two or more needles along the longitudinal axis of the shaft within the inner shaft.

7. The device of claim 1, wherein:
the first slider mechanism defines a bi-leveled groove;
the second slider mechanism comprises a post positioned within the bi-leveled groove; and
mechanical engagement between the bi-leveled groove and the post prevents movement of the first slider mechanism when the two or more needles are in the second position.

8. The device of claim 1, wherein the two or more needles can be actuated to emerge radially from the shaft at multiple positions along the longitudinal axis of the shaft.

9. The device of claim 1, wherein the device further comprises an indicator that shows a position of the two or more needles along the longitudinal axis of the shaft.

10. The device of claim 1, wherein the shaft has a measuring scale proximal of the two or more needles.

11. The device of claim 1, further comprising an actuation indicator that indicates whether the needles are in the first position or the second position.

12. The device of claim 11, wherein the actuation indicator is positioned along the handle.

13. The device of claim 11, wherein the actuation indicator is positioned on a safety switch that is configured to cover the second slider mechanism.

14. The device of claim 1, further comprising a treatment fluid reservoir configured to distribute treatment fluid between the two or more needles.

15. A method of treating urinary incontinence, the method comprising:
inserting a shaft of an injection device into a urethra so that a distal tip portion of the shaft resides within a bladder;
positioning a meatus clamp of the injection device against a urethral meatus of the urethra;
after positioning the meatus clamp, measuring a length of the urethra using a scale on the shaft;
sliding a first slider mechanism along a longitudinal axis of the shaft to cause two or more needles to move longitudinally along the shaft;
sliding a second slider mechanism distally along the shaft to cause the two or more needles to extend radially from the shaft so that a distal tip portion of each of the two or more needles puncture and extend through an inner wall of the urethra; and
while the two or more needles are extending through the inner wall of the urethra, injecting a therapeutic via the two or more needles.

16. The method of claim 15, further comprising:
expanding an expandable member that is attached to the distal tip portion of the shaft; and
applying proximal traction of the shaft to cause the expandable member to abut an inner wall of the bladder around an opening to the urethra, wherein the length of the urethra is measured while the proximal traction is being applied.

17. The method of claim 15, wherein: sliding the second slider mechanism distally along the shaft to cause the two or more needles to extend radially from the shaft comprises:
raising a safety switch to expose the second slider mechanism;
sliding the second slider mechanism distally to an actuated position in which a portion of the second slider mechanism manually engages a portion of the first slider mechanism to prevent movement of the first slider mechanism; and
lowering the safety switch over the second slider mechanism.

18. The method of claim 15, further comprising:
after injecting the therapeutic, sliding the second slider mechanism proximally along the longitudinal axis of the shaft so that the distal tip portion of each of the two or more needles withdraws inside the shaft.

19. The method of claim 15, wherein the two or more needles are moved longitudinally along the shaft and multiple injections are delivered at multiple locations longitudinally along the urethra.

20. The method of claim 15, wherein the therapeutic comprises exosomes or platelet lysate.
